(19) United States
(12) Patent Application Publication
(10) Pub. No.: US 2011/0040383 A1
(43) Pub. Date: Feb. 17, 2011

(54) SPINAL COLUMN IMPLANT

(76) Inventor: Corinna Wurfel, Filderstadt (DE)

Correspondence Address: DAVID A. GUERRA;INTERNATIONAL PATENT GROUP, LLC, Suite 700, 1816 Crowchild Trail N.W., CALGARY, AB, T2M 3Y7, CA

(21) Appl. No.: 12/988,662

(22) PCT Filed: Apr. 25, 2009
(86) PCT No.: PCT/DE09/00592

§ 371 Date: Oct. 20, 2010

(51) Int. Cl. A61F 2/44

(52) U.S. Cl. 623/17.11

(57) ABSTRACT

1. The invention relates to a spinal column implant in veterinary- or human medicine for stabilizing of spinal column segments. 2.1 Known implants of this kind which are installed between the spinous processes for stabilizing of spinal column segments, require a medial access and the re-section of the ligamentum supraspinale, which can have a deleterious effect on the ligament and on wound healing. 2.2 The implant according to the invention features a stirrup whose free ends run in the direction of the spinous processes. The free ends are provided with means to rest against the spinous processes, wherein at least one of the means is designed as a saddle-like support. The width of the implant can be reduced for implantation so that a lateral implantation between the spinous processes is possible without destruction of the ligamentum supraspinale.

The invention relates to a spinal column implant in veterinary or human medicine, for stabilising (also dynamically) spinal column segments, with an essentially U-shaped stirrup, which can be inserted between two spinous processes, the free ends of which run in the direction of the spinous processes, with means on the free ends for supporting on the spinous processes.

Implants in human medicine are known which are inserted between two adjacent spinous processes of the spinal column in order to stabilise the vertebra, for example in the case of degenerative changes in the intervertebral disc and to counteract hypermobility of the vertebra. Similar implants for use in veterinary medicine are not known.

A further indication for such an implant is “kissing spine syndrome”, which can occur in horses. In this case the spinous processes are in contact with each other and painfully grind each other down, which can result in the horse being unable to be ridden.

Kissing spine syndrome is a common disorder in horses for which there some treatment approaches, whereby so far surgical procedures are limited to resection of the spinous processes.

Spondylarthrosis is a common disease in dogs. Dynamic and mechanical factors play a decisive role in its development. Spondylarthrosis is a primary degenerative disease in which partial tissue ruptures, particularly in the area of the intervertebral ligaments occur through compression, tensile and shearing movements on the support and ligament system. These microtraumas lead to fibre proliferation in the periostium of the ventral spinal column and anterior longitudinal ligament. Along the fibres exostoses occur which increase in size and grow towards each other. They can fuse to form a solid osseous plate (bamboo spine). The bone proliferation partially extends to the lateral aspects of the spinal column and can lead to osseous narrowing of the nerve exit points which is associated with severe pain and symptoms of paralysis.

Implants in human medicine for the stabilisation and/or distraction of vertebral bodies are known (e.g. U.S. Pat. Nos. 5,645,599, 5,860,977, 5,496,318). The area of application of these implants extends to the treatment of diseases of the spinal column in humans, e.g. of degenerated discs or narrowing of the spinal canal (stenoses). These implants are inserted between the spinous processes either laterally or from posterior.

Implants for posterior insertion required the removal of the supraspinous ligament as well as complete resection of the intraspinal ligament, whereas implants that are inserted laterally only require the less traumatic removal of part of the intraspinal ligament. Implants of both types are secured against lateral migration by means of lateral projections or with synthetic straps surrounding the spinous processes.

Another differentiating feature in addition to insertion and fixing is the elasticity of the known implants, which is divided into angular elasticity and axial compressibility. Whereas the majority of laterally implantable implants exhibit no significant compressibility, some of the implants for posterior insertion are provided with a spring elastic element (U.S. Pat. No. 5,645,599, EP-A-1 054 638). It can be assumed that a spring elastic implant allows considerably more physiological movement than a rigid spacer. Taking into consideration the angular elasticity and compressibility, very rigid implants without angular elasticity (e.g. U.S. Pat. No. 5,860,977), medium-rigid implants with physiological angular stability (e.g. U.S. Pat. No. 5,645,599) and relatively soft implants with low compression or angle change stability are known (e.g. DE 699 13 659).

For the successful treatment of the above conditions an intraspinously acting implant must be able to be inserted laterally. It should exhibit distraction-adapted rigidity in the axial direction, as well a physiologically effective angular stability in order to counteract hypermobility in the treated segment. On the other hand restriction of flexion movements by the implant is general not desirable, as this considerably hinders the physiological scope of movement which can result in overloading of the posterior structures.

The aim of the present invention is to stabilise hypermobile spinal column segment by means of an elastic implant without significantly restricting the range of movement of the vertebral bodies. In dogs it is important that the ligament apparatus is impaired as little as possible by the surgery in order to counteract any further destabilisation. In horses the decisive factor is permanently preventing contact between the spinous processes in that the implant acts as a spacer. As with dogs, minimising ligament apparatus trauma with as small surgical access as possible is of great advantage for rapid healing and rehabilitation, as in horses the disease mainly occurs in the saddle area and horses tend to excessive scar formation.

In summary this means combining the physiological advantages of spring-elastic implants for insertion from posterior with the advantages of latterly inserted, less traumatic implant designs.

This aim is achieved in accordance with the invention by means of an implant of the type described in the introduction in that at least one of the means of support on the spinous process is designed as a saddle-like support. Further features are set out in the sub-claims.

The present invention relates to an angular elastic implant with at least one anatomically-shaped, saddle-like support surface on the spinous processes. Through dispensing with a second pair of wings (as, for example, described in U.S. Pat. No. 5,645,599) the essentially U-shaped implant allows unrestricted flexion movement of the spinal column and can at the same time be laterally inserted. One variant of the implant also allows rotation movements to a physiological extent whereby the centre of rotation defined by the implant corresponds with the centre of rotation of the vertebral segment.

During the course of a disease instability of the spinal column can trigger painful spondylarthrosis in dogs, which can be treated by an implant in accordance with the invention.

Restoration of a space between the spinous processes involved in kissing spine syndrome by means of the implant in accordance with the invention appears to very promising as a possible alternative treatment approach in horses.

In addition, the implant forming the basis of the invention is suitable for stabilising the spinal column in the field of human medicine.

The application of supports for an insertion instrument which can hold the implant in a compressed position, simplifies lateral insertion into the space between the spinous processes. Due to the high level of angular flexibility the implant allows a significant reduction in height so that the implant can be inserted laterally. Through appropriate structural design (broadening, thickening of the wall, insertion of an additional reinforcement clip) of the ventral aspect of the implant a large spreading effect can be achieved without essentially restricting the angular flexibility. In an alternative variant of the implant lateral insertion takes place through pushing the lower part of the implant into the upper part, which comprises two shells. Through the subsequent introduction of an elastic strand into the intermediate space between the upper shells, movement damping is achieved and at the same time the required spreading effect can be set.

Fixing between the spinous processes can take place either by way of a clip-like holder applied to a shank of the implant, or by means of a loop or retaining strap, made of synthetic material for example. The loops or retaining straps can be passed around the spinous process or laterally past the vertebral arch (in a sublaminar fashion). Fastening of the implant on the vertebral arch can also take place with clips which surround the vertebral arch from dorsal.

In order to treat hypermobility in the area of the last lumbar vertebra and first sacral vertebra the anatomical conditions require special types of fastening. As the spinous process of the first sacral vertebra is subject to large individual variation in size, this can only be used for fastening the implant in isolated cases. However, by means of lateral spurs on its lower shank the implant can be fastened through the forr. sacralia dorsalia with loops or retaining straps or by way of clips which also use the forr. sacralia dorsalia for anchoring. Another attachment option is constituted by lateral spurs of the implant which after prior preparation are pushed between the sacrum and tendons attached to it.

This achieves good anchoring and at the same time through stimulation of the spurs on the anatomical structures connective tissue stabilisation thereof can be achieved. The fibrosing can even extend so far that slight shortening of the structures occurs whereby they draw together the vertebral sections dorsally and thereby raise them ventrally.

Expediently the implant is made of a material which allows a high degree of elastic deformation of the implant during its implantation. Examples of such materials, but not restricted thereto are nitinol, titanium and/or titanium alloys, spring or implant steel, synthetics materials such as, for example, polyethylene, polycarbonate urethane, PEEK, PEK, PEKK, PSU, PPS, or silicone, as well as fibre composite material or combinations of several of these materials.

Due to a physiologically adapted stability of the implant a reduction in hypermobility of a segment treated with it is possible and a positive influence on a spondylarthrosis-related disease process in dogs is likely.

In horses the lateral insertion of the implant between the spinous processes largely allows the ligament system to be preserved. The comparatively small surgical access possible with the implant minimises the otherwise known problems of excessive scar formation in the saddle area.

Applications in the field of human medicine are improved through the less traumatic implantation technique of the implant in accordance with the invention.

Other advantages, features and details of the invention are set out in the following description which sets out several examples of embodiment in more detail with reference to the drawings. The features shown in the drawings as well as set out in the claims and description can each be essential to the invention individually or in any combination.

In the drawing

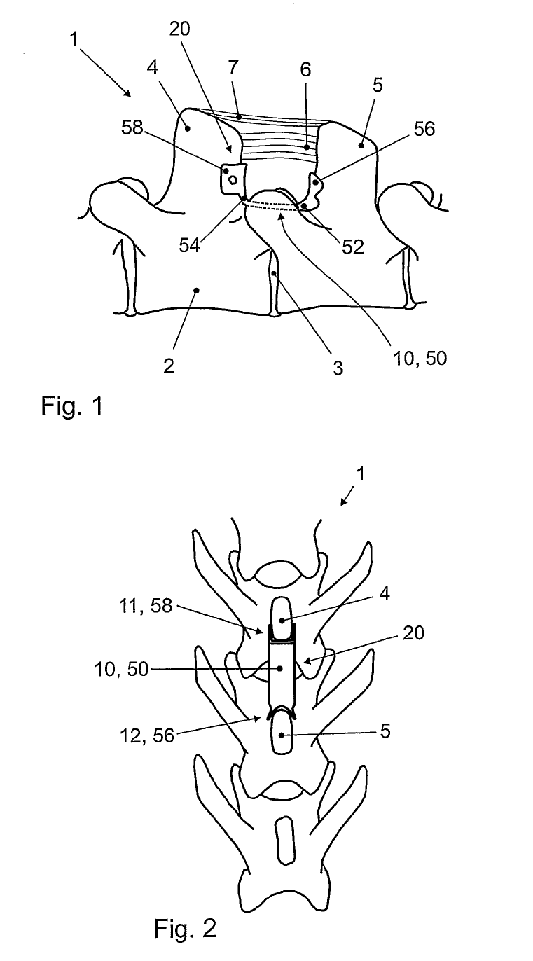

FIG. 1: Shows a lateral view of the inserted implant

FIG. 2: Shows a dorsal view of the inserted implant

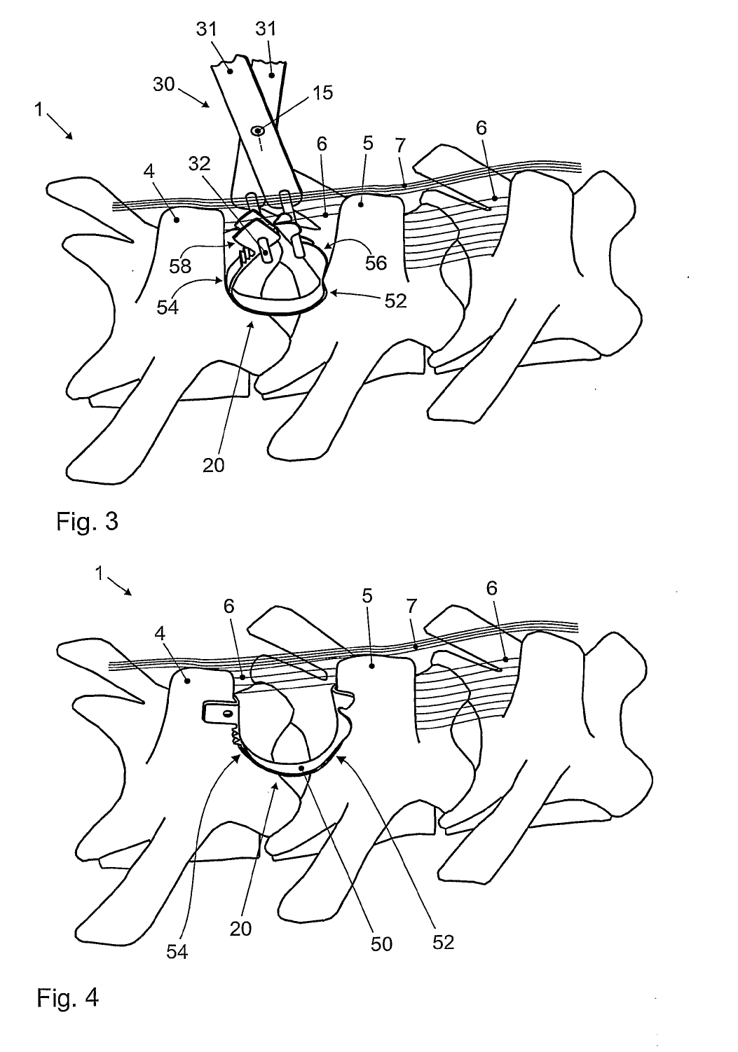

FIG. 3: Shows a perspective view of the implantation

FIG. 4: Shows a perspective view of the inserted implant

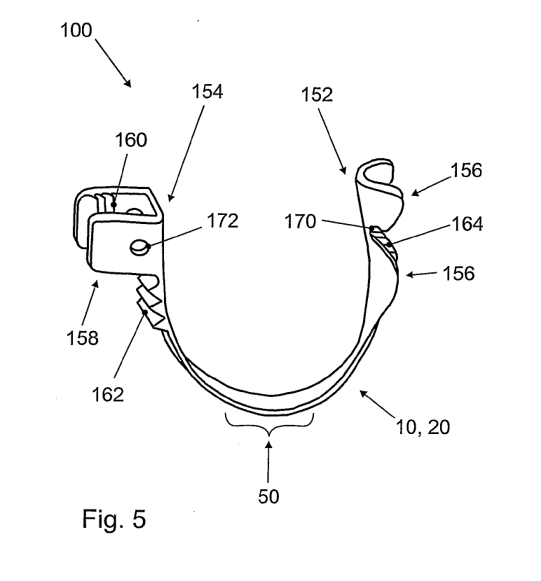

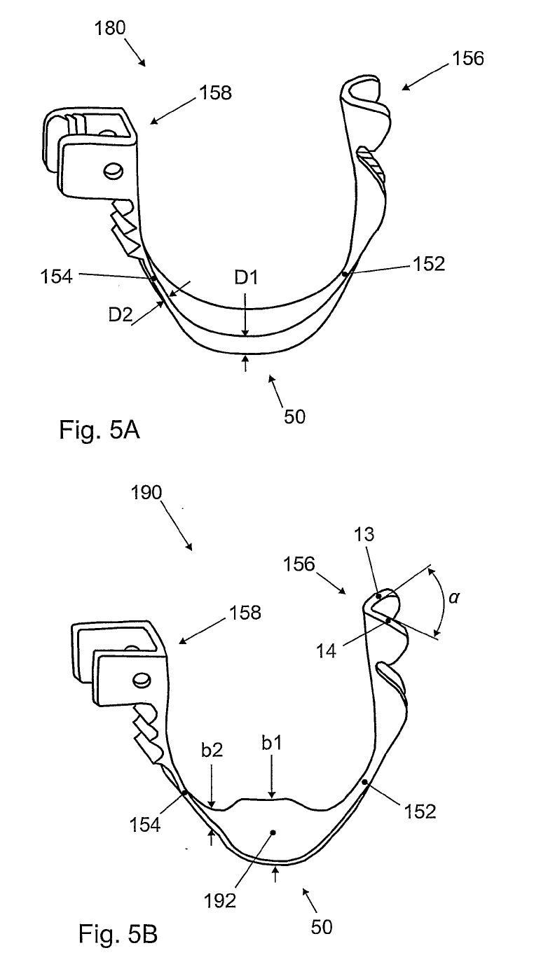

FIG. 5: Shows a detailed view of the implant

FIG. 5A: Shows a first variation of the implant in accordance with FIG. 5

FIG. 5B: Shows a second variation of the implant in accordance with FIG. 5

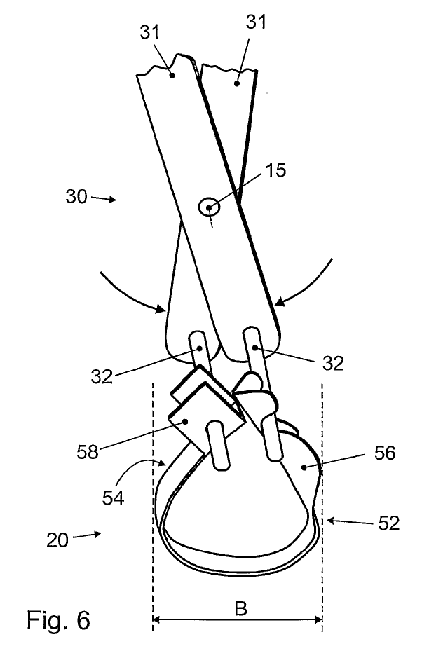

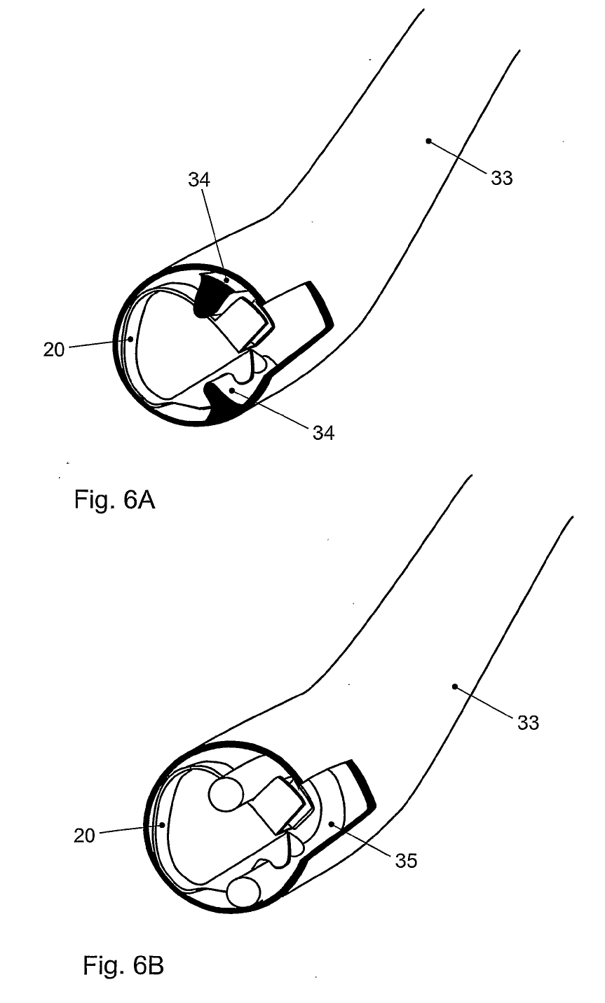

FIG. 6: Shows the implant compressed by tongs

FIG. 6A: Shows an implant which can be implanted with an implanting aid (sleeve)

FIG. 6B: Shows an implant with a compression clasp which can be implanted with an implanting aid (sleeve)

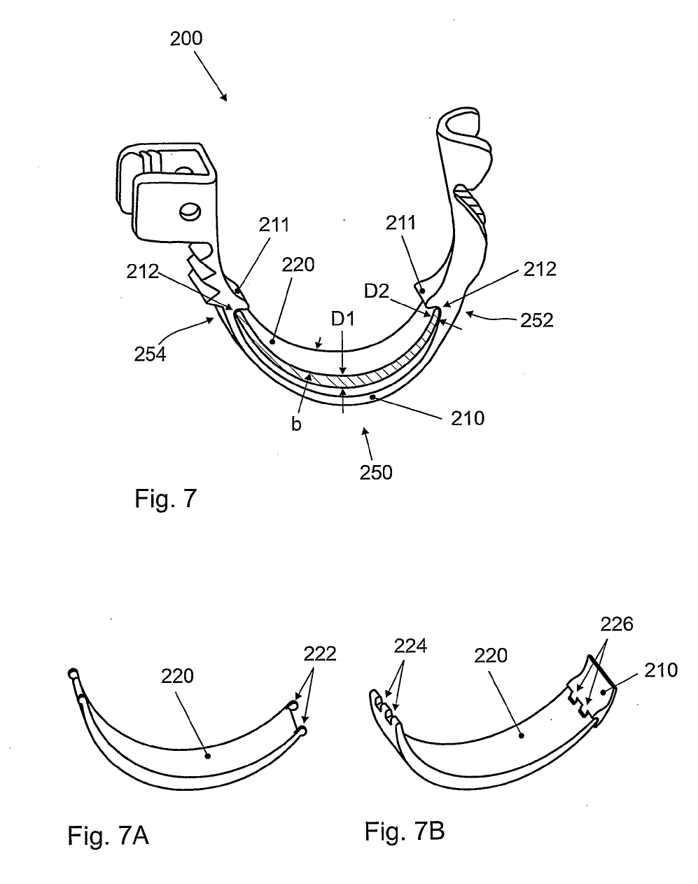

FIG. 7: Shows a view of the implant with a ventral stabilisation clip

FIG. 7A: Shows a detailed view of a first embodiment of the stabilisation clip in accordance with FIG. 7

FIG. 7B: Shows a detailed view of a second embodiment of the stabilisation clip in accordance with FIG. 7

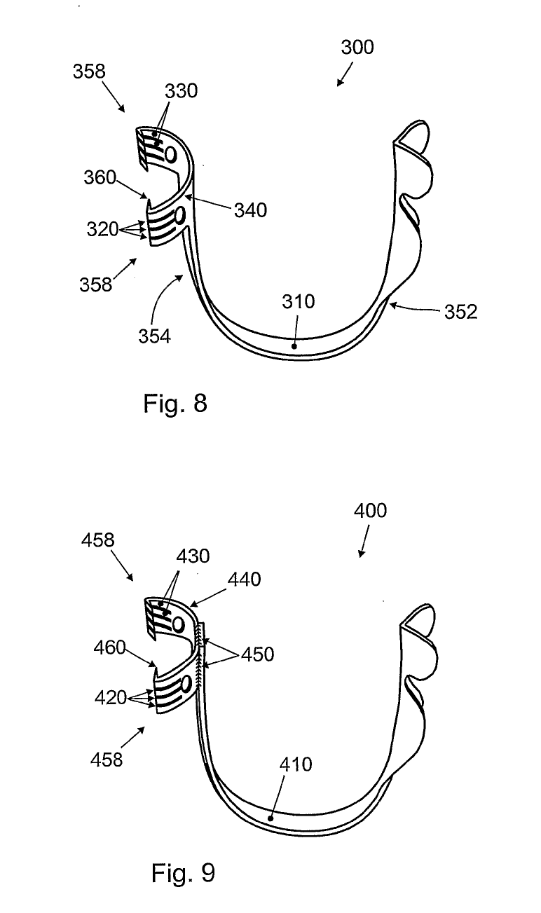

FIG. 8: Shows a variant of the implant with optimised support on a spinous process

FIG. 9: Shows a variant of the implant according to FIG. 8 made of two different materials

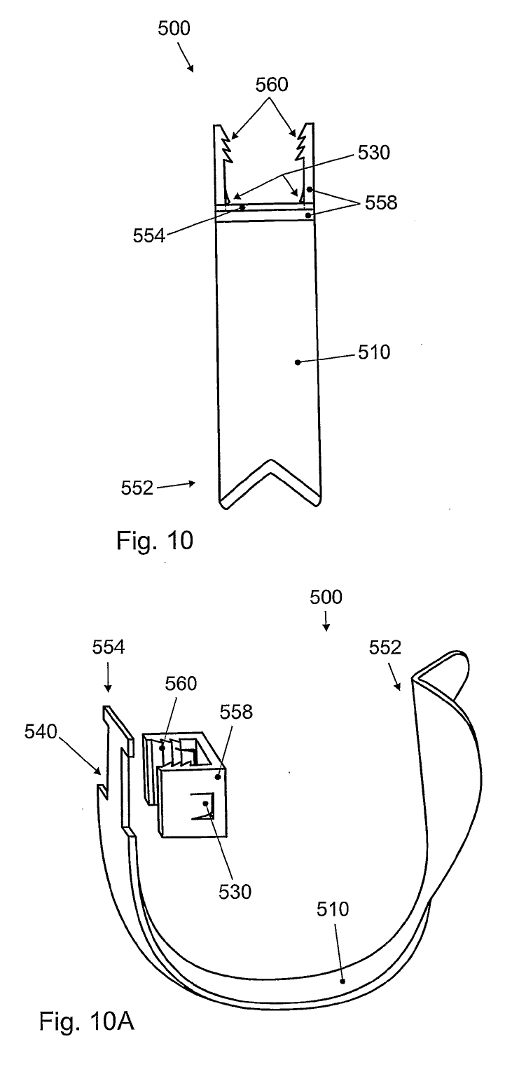

FIG. 10: Show a dorsal view of a further two-part variant of the implant

FIG. 10A Shows an exploded view of the implant in accordance with FIG. 10

FIG. 11 Shows a dorsal view of a further three-part variant of the implant

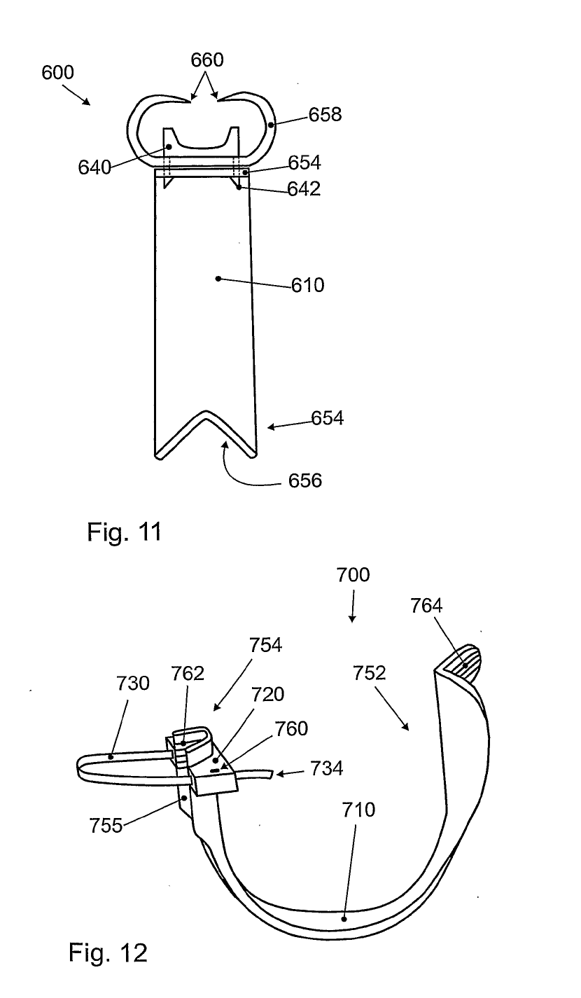

FIG. 12 Shows a further variant of the implant with retaining straps for attachment to the spinous process

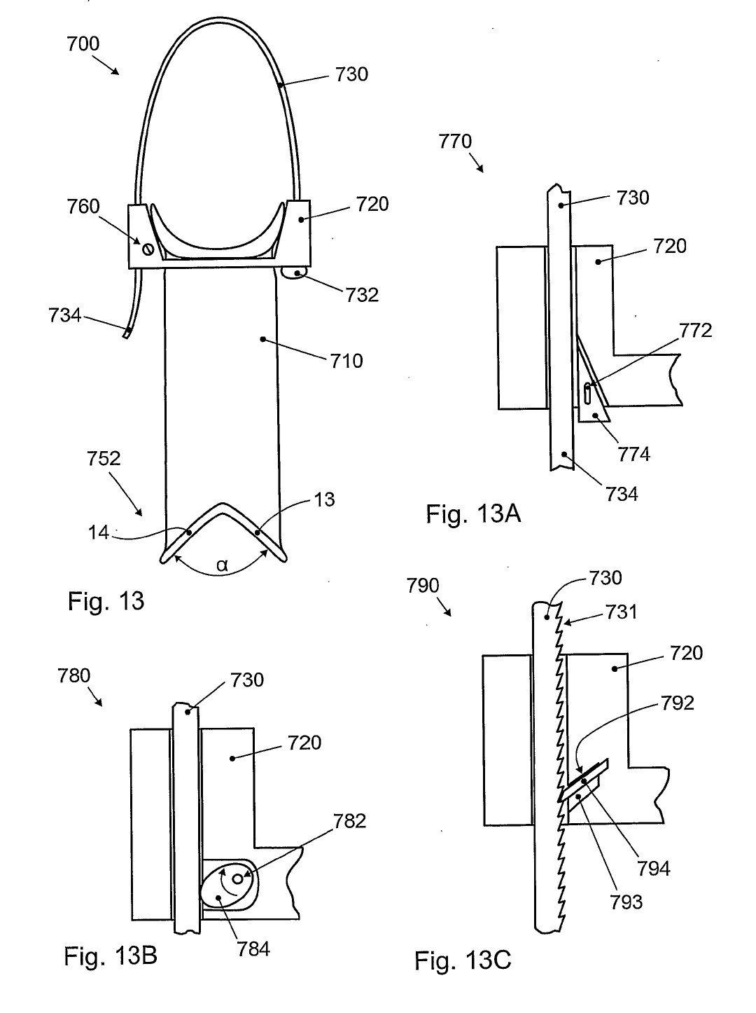

FIG. 13 Shows a dorsal view of the implant in accordance with FIG. 12

FIG. 13A Shows a first attachment variant of the retaining strap on the implant

FIG. 13B Shows a second attachment variant of the retaining strap on the implant

FIG. 13C Shows a third attachment variant of the retaining strap on the implant

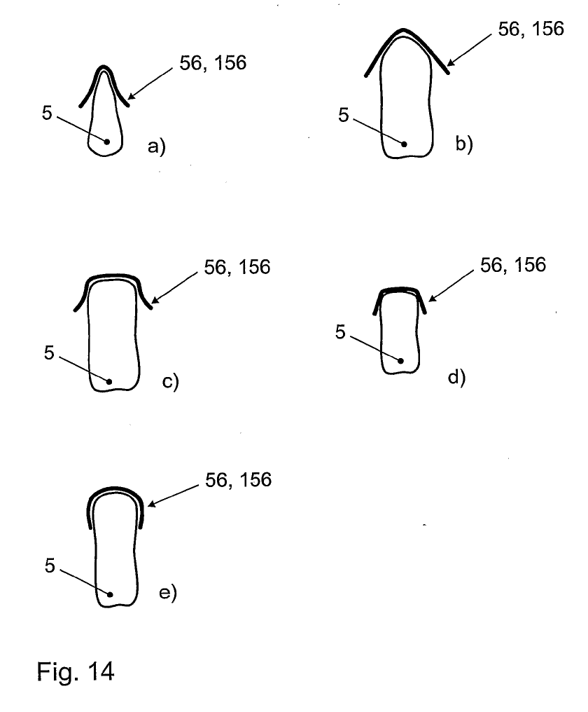

FIG. 14: Variants a) to e) of the shape of the saddle-like support

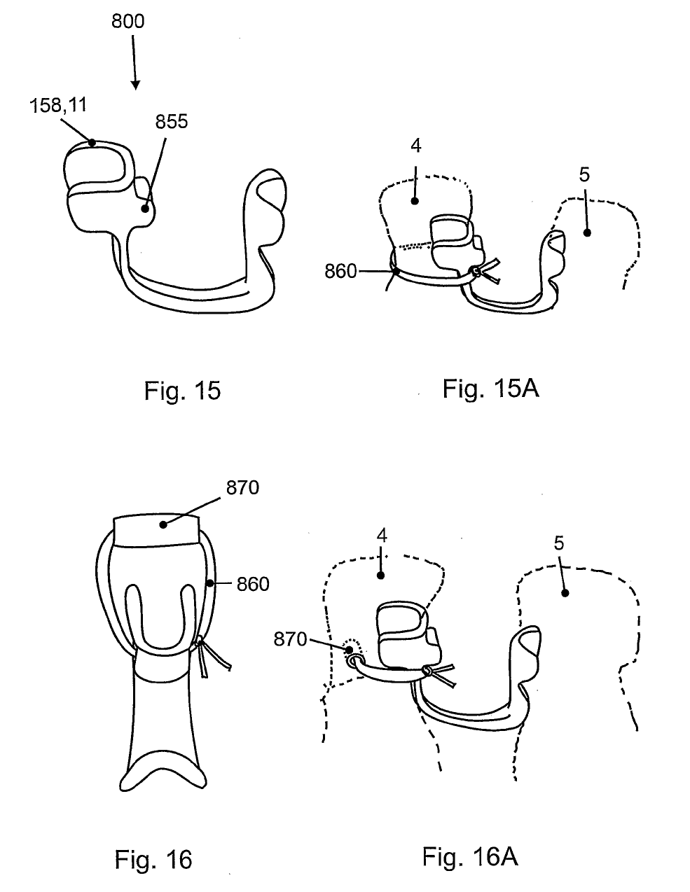

FIG. 15: Shows a perspective view of a further implant variant with possibility of attachment of a retaining strap

FIG. 15a: Shows the implant in accordance with FIG. 15 after implantation with the retaining strap around the spinous process

FIG. 16: Attachment variant of FIG. 15a: dorsal view of the implant with retaining strap and sleeve for the spinous process

FIG. 16a: Shows the implant in accordance with FIG. 16 after implantation and insertion of the sleeve into the spinous process

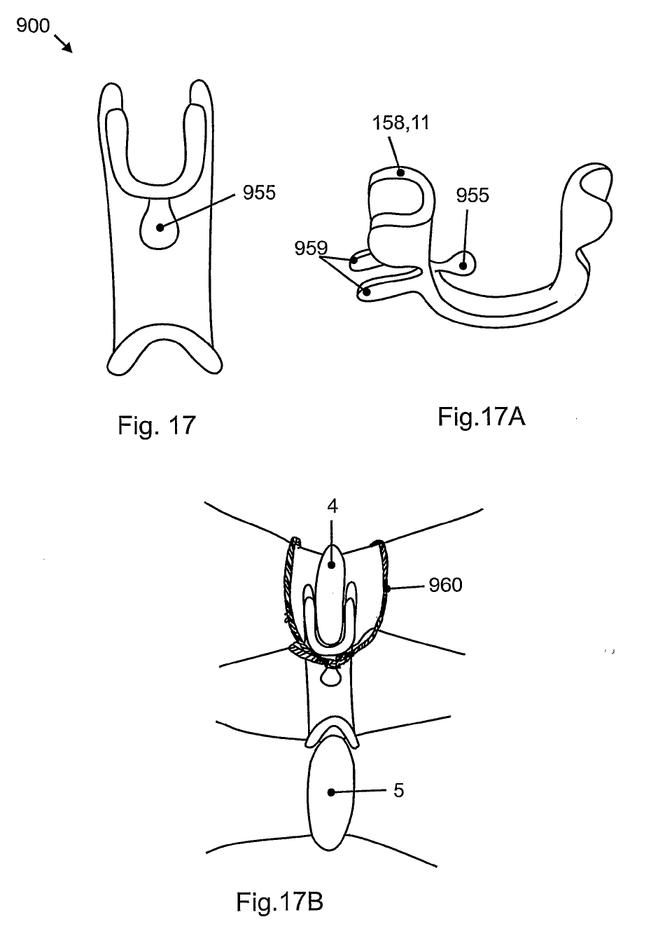

FIG. 17: Shows a dorsal view of a variant of the implant with fastening means for loops/retaining straps

FIG. 17a: Perspective view of the implant in accordance with FIG. 17

FIG. 17b: Dorsal view of the implant in accordance with FIG. 17 after implantation and fixing with retaining straps around the vertebral arch

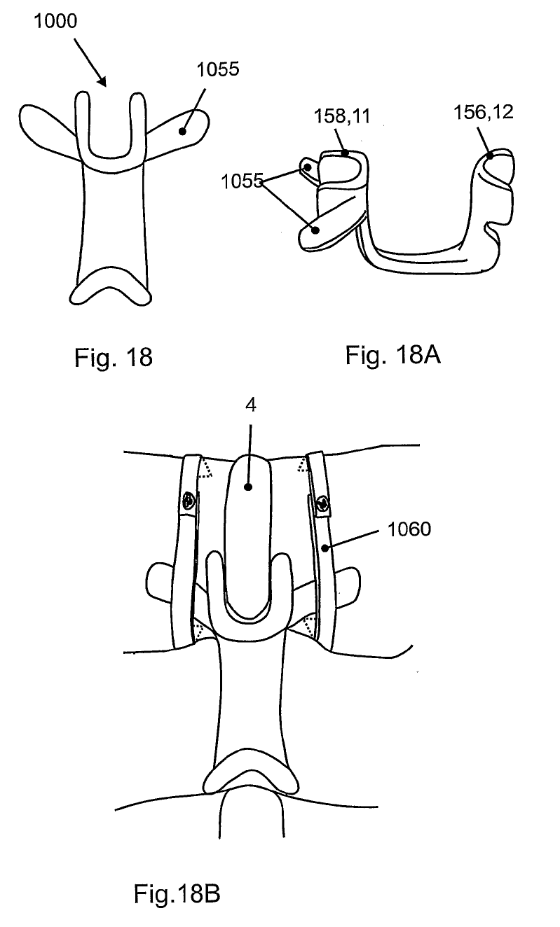

FIG. 18: Shows a dorsal view of a further variant of the implant with lateral projections

FIG. 18a: Shows a perspective view of the implant in accordance with FIG. 18

FIG. 18b: Shows a variant of the implant in accordance with FIG. 18 after implantation and fixing to the vertebral arch with clips

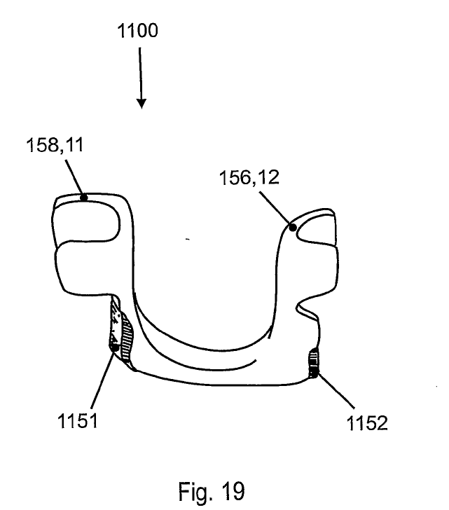

FIG. 19: Shows a perspective view of a further variant of the implant with cushioned support surfaces for the laminas

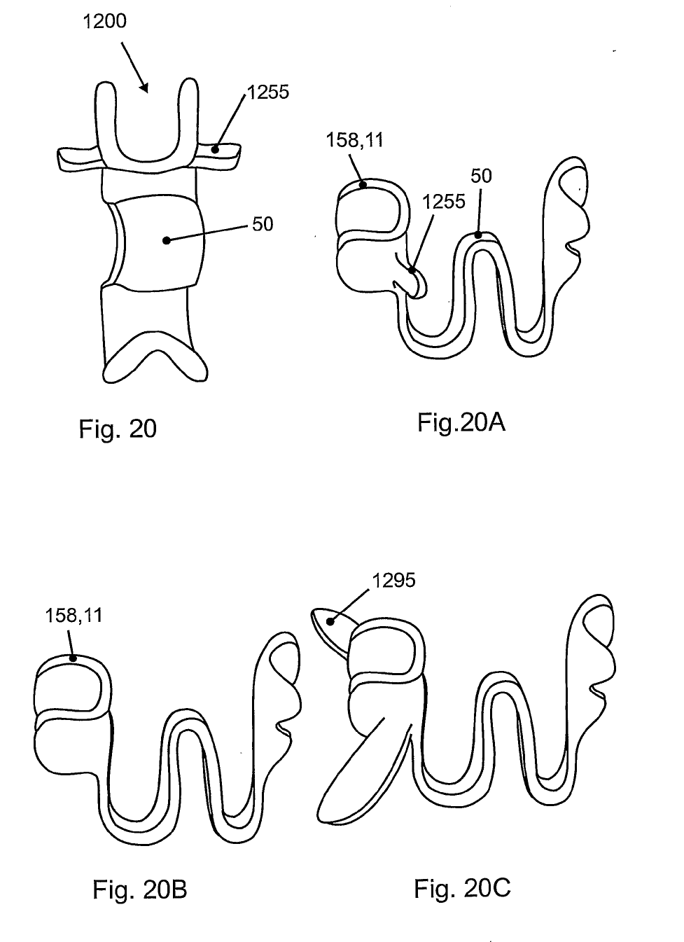

FIG. 20: Shows a dorsal view of a further variant of the implant with triple curved ventral aspect

FIG. 20a: Shows a perspective view of the variant of the implant in accordance with FIG. 20

FIG. 20b: Shows a modification of the implant in accordance with FIG. 20 without lateral fastenings for loops, straps or clips

FIG. 20c: Shows a further modification of the implant in accordance with FIG. 20 with lateral wings for fastening to the vertebral arch by means of clips

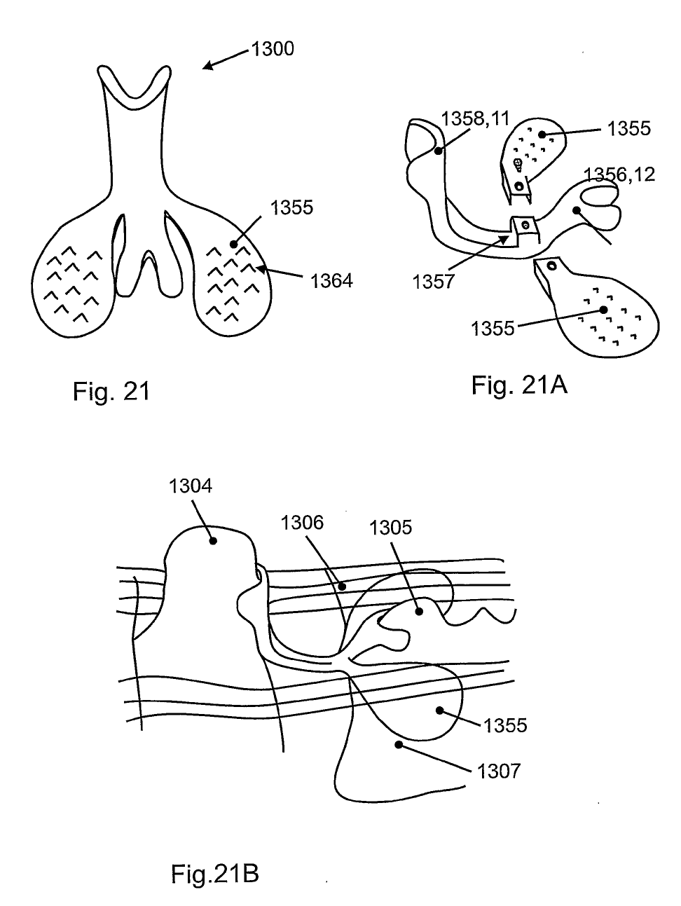

FIG. 21: Shows a further variant of the implant for the area last lumber vertebra—first sacral vertebra with spurs

FIG. 21a Show a perspective exploded view of the implant in accordance with FIG. 20 with subsequently attachable spurs

FIG. 21b Shows the implant in accordance with FIG. 21 after implantation

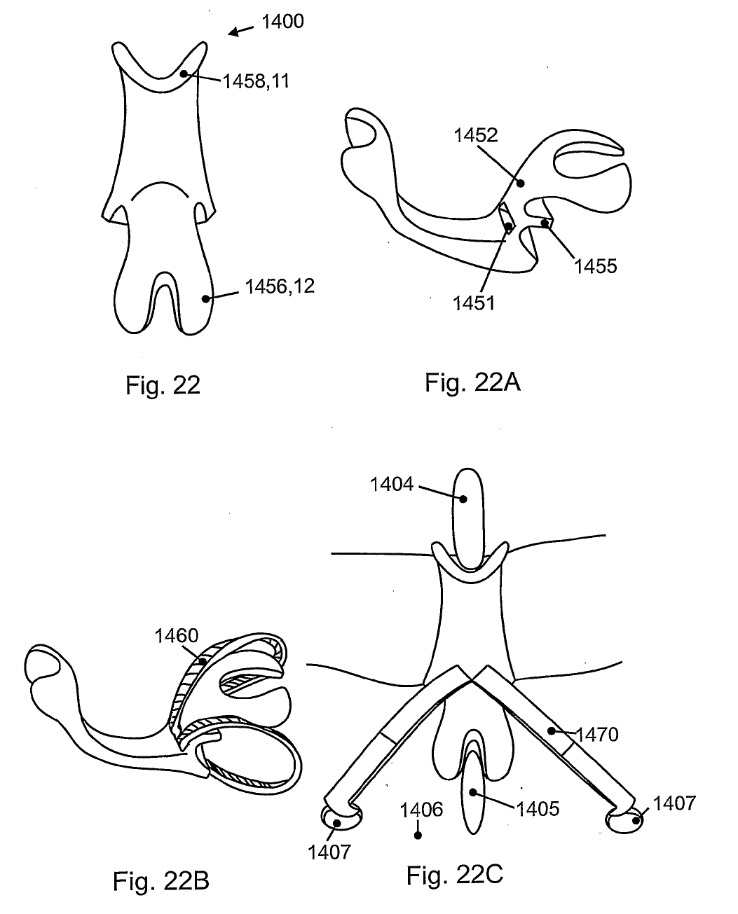

FIG. 22 Shows a dorsal view of a further variant of the implant for the area last lumbar vertebra—first sacral vertebra

FIG. 22a Shows a perspective view of the implant in accordance with FIG. 22

FIG. 22b Shows the implant in accordance with FIG. 22 with fastening means/loops on the sacrum

FIG. 22c Shows a dorsal view of the implant in accordance with FIG. 22 fixed with clips after implantation

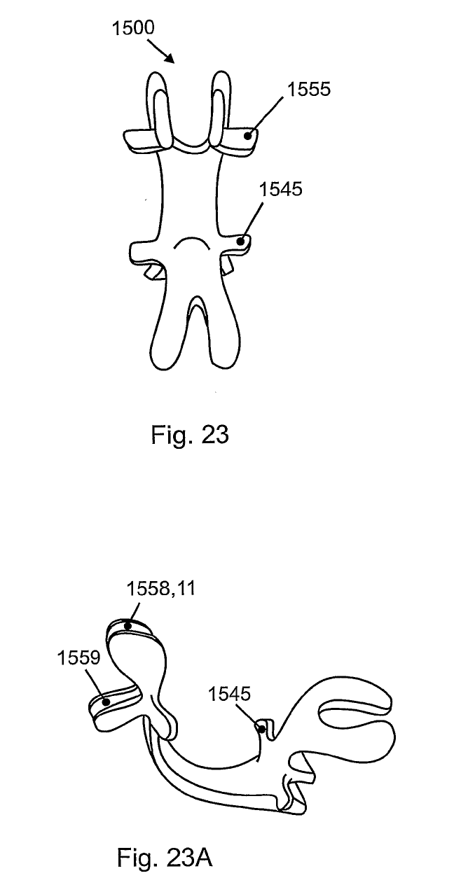

FIG. 23 Shows a dorsal view of a further variant of the implant

FIG. 23a Show a perspective view of the implant in accordance with claim 23

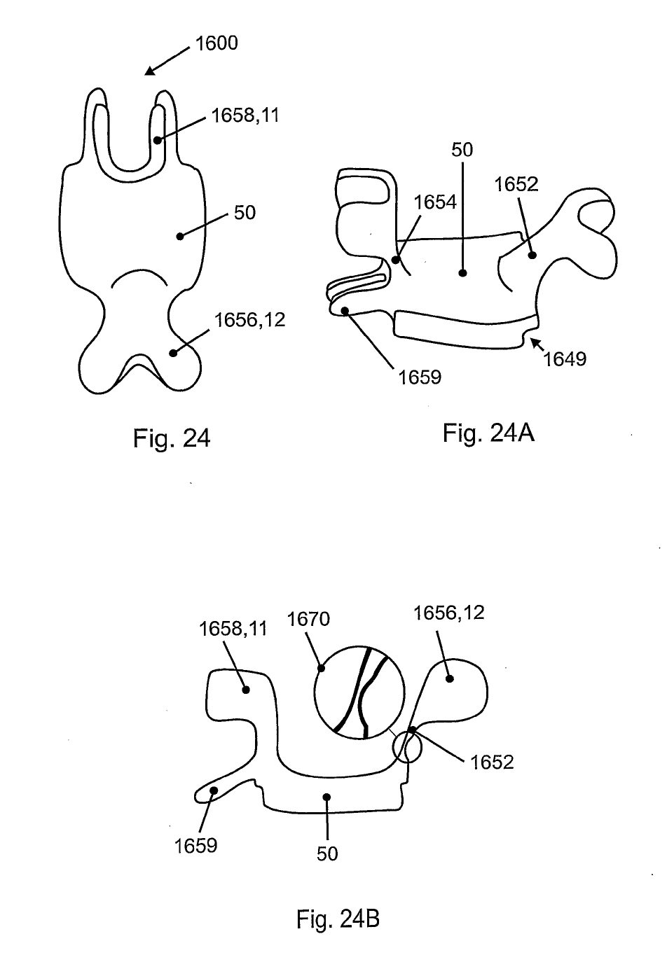

FIG. 24 Shows a dorsal view of a further variant of the implant

FIG. 24a Shows a perspective view of the implant in accordance with FIG. 24

FIG. 24b Shows a side view of the implant in accordance with FIG. 24 with a detailed section

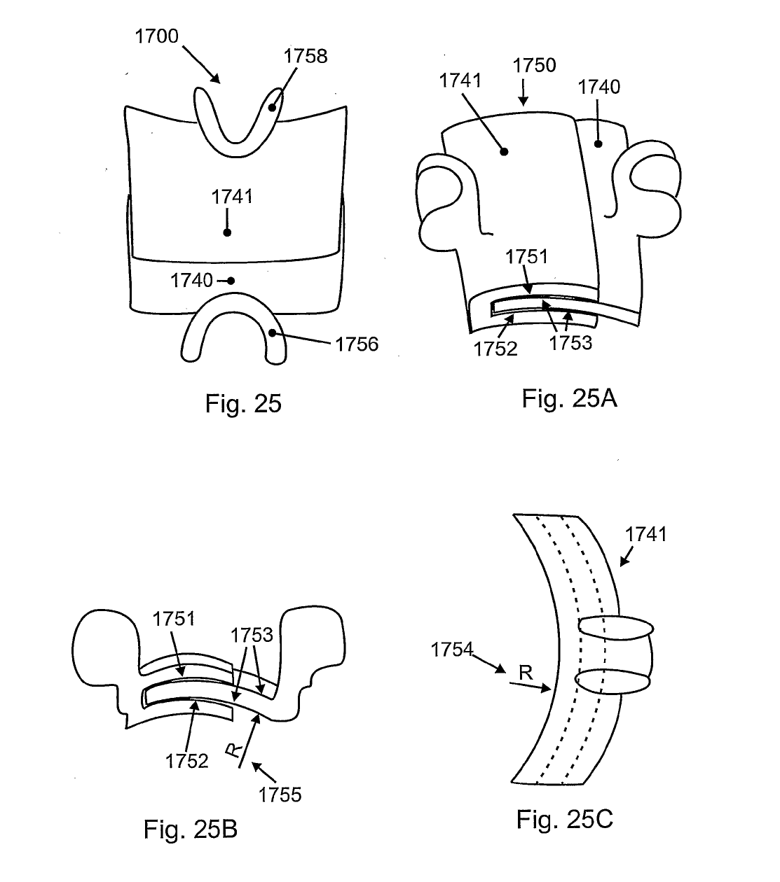

FIG. 25 Shows a dorsal view of a further variation of the implant

FIG. 25a Shows a perspective view of the implant in accordance with FIG. 25

FIG. 25b Shows the implant in accordance with FIG. 25 from the side

FIG. 25c Shows the implant in accordance with FIG. 25 from above

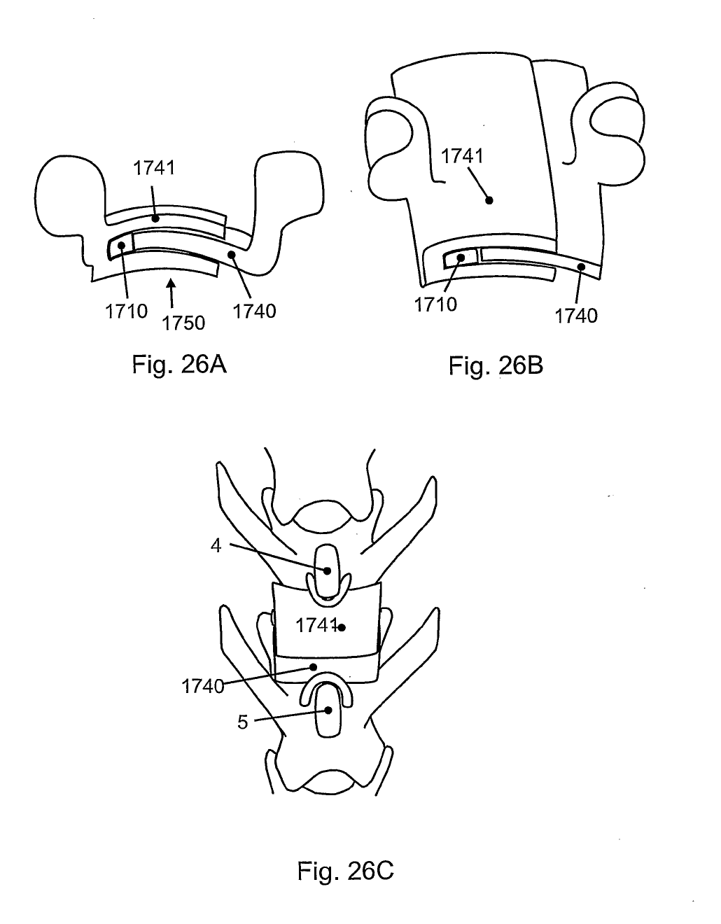

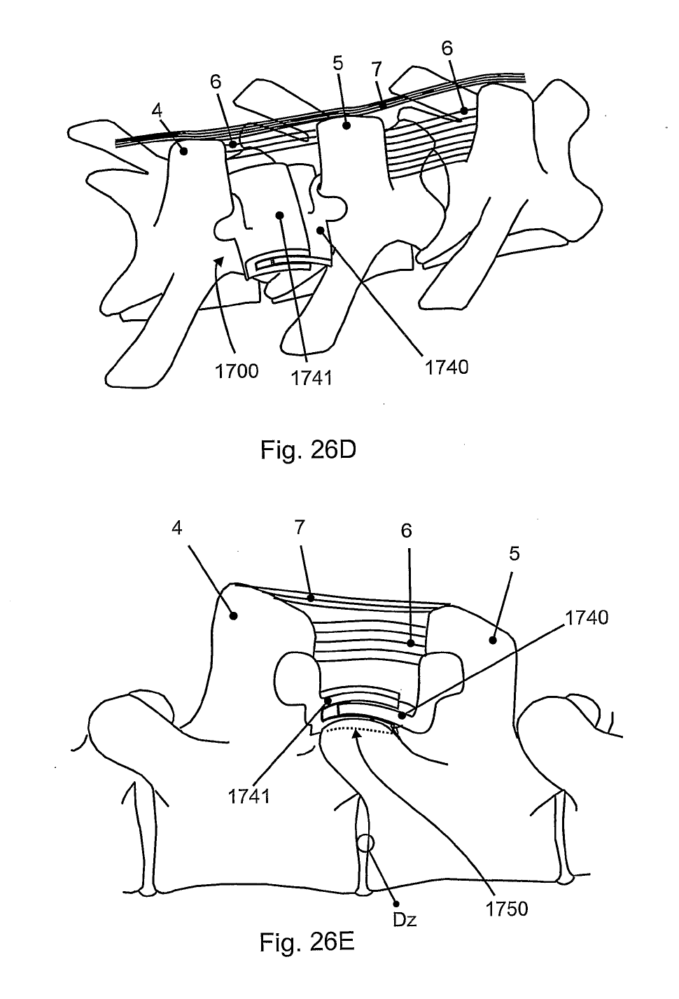

FIG. 26a Shows a front view of a variant of the implant in accordance with FIG. 25

FIG. 26b Shows a perspective view of the implant in accordance with FIG. 26a

FIG. 26c Shows a dorsal view of the implant in accordance with FIG. 26a after implantation

FIG. 26d Shows a perspective view of the implant in accordance with FIG. 26a after implantation

FIG. 26e Shows a side view of the implant in accordance with FIG. 26a after implantation

FIG. 1 shows, inserted into a spinal column segment 1, the spinal column implant 20 which is positioned between two adjacent vertebral bodies 2, more particularly between two spinous processes 4 and 5. Above and between the spinous processes 4 and 5 extend the suprapsinal ligament 7 and the interspinal ligament 6, which are largely spared through the lateral implantation. The implant 20, which is essentially in the form of a stirrup 10 is supported on the spinous processes 4 and 5.

FIG. 2 shows a dorsal view of the inserted implant 20, whereby means 11 and 12, connected to each other on the ventral aspect 50, can be seen supported on the spinous processes 4 and 5. In FIG. 1 it can also be seen that the implant 20 is supported on one spinous process 4 by means of a U-shaped shoe 58 and on the other spinous process 5 by means of a saddle-like support 56. The cross-section of the shoe 58 and the support 56 can be seen in FIG. 2.

FIG. 3 shows the insertion of the implant 20 between the spinous processes 4 and 5 of a spinal column, of a dog for example. The implant 20 has two shanks 52 and 54 which are compressed with a tongs-like compression instrument 30 so that the maximum breadth B of the implant 20, measured from the saddle-like support 56 to the circumferential edge of the shoe 58 is considerably reduced and lateral insertion (after prior removal of part of the interspinal ligament 6) is possible. The compression instrument 30 has two tools 32 arranged laterally on the tong arms 31, more particularly pins, hooks or projections protruding parallel to the direction of the axis 15 of the instrument, which engage at a suitable point on the implant 20 and allow compression and lateral insertion of the implant 20 between the spinous processes 4 and 5.

After discontinuing the compression and removal of the compression instrument 30, as shown in FIG. 4, the implant is supported on the spinous processes 4 and 5 by the restoring force of the spring-elastic connection 50 and the also spring-elastic shanks 52 and 54.

FIG. 5 shows a first specific embodiment 100 of the implant 20 in accordance with the invention, with an elastic connection 50 on the ventral aspect of the implant which connects the first elastic shank 152 with the second elastic shank 154.

Together with the elastic connection 50 the shanks 152 and 154 form the U-shaped stirrup 10. For supporting on the spinous process 5 the implant 20 has a saddle-shaped support 156.

With extreme flexion of the spinal column 1 this saddle-like support 156 allows the implant 20 to be lifted from the spinous process 5 thereby permitting unhindered flexion movement. Suitable implant recesses 170 and 172 serve to hold the compression instrument 30. Suitable teeth or surface modifications 162 and 162, which can also be in the form of a rough section or a coating, fix the implant 20 in the dorsoventral direction.

For attaching to the spinous process 4 there is a clip-like shoe 158 which on its inner surface has suitable surface modifications 160, more particularly teeth.

FIG. 5A shows a first variant 180 of the implant 20, which in the ventral aspect 50 can vary in thickness D. Thus the middle section 162 can be thicker than the transition to the shanks 152, 154 of the implant 20 (D1>D2) in order to achieve greater stability in the ventral aspect 50 of the implant 20.

FIG. 5B shows a second variant 190 of the implant 20 in which the breadth b of the ventral aspect varies. Thus the middle section 192 of the aspect 50 can be broader than the transition to the shanks 152, 154 of the implant (b1>b2).

In FIG. 5B the angle a between the support shanks 13 and 14 of the saddle-like support 156 is shown. Depending on the design of the saddle this angle α can be adapted to the anatomical situation of the species in question and can assume values of between 0° and 150°, preferably between 10° and 90°. This can be seen in more detail in the dorsal view which will be described below. The angle can also be adapted to the anatomy. The support shanks 12 and 14 are not even but adapted to the anatomical shaped of the lamina and spinous process in that they start slightly curved or U-shaped and then in a first angle become V-shaped, which then progresses into another, second angle. In order to facilitate lateral insertion it is also possible for support shank 14 to be shorter than the other shank 13.

In preferred examples of embodiment the angle α depends on the species and race of the animal. As a rule the saddle is only V-shaped in the broadest sense and has a relatively rounded shaped instead of a smooth, sharp-edged angle. Instead of soft V-shape a U-shape is also possible for the support, whereby the shanks are open between 0° and 30° close to where they are connected, and then diverge up to 90° towards the end of the shank, e.g. by way of a second, soft kink or rounded shape.

FIG. 6 shows a detailed view of the implant 20 compressed with the compression instrument 30. Through the tools 32 arranged laterally on the tong arms 31 the implant 20 is compressed in the area of the flexible shanks 52, 54 in such a way that a considerable reduction in breadth takes place. This allows lateral implantation as the breadth of the implant 20 is reduced by around the extent of the shoe 58 and support 56 to the nominal implant breadth B.

FIGS. 6A and 6B show the implant 20 in the compressed state within an implanting sleeve 33. The implanting sleeve 33 allows minimally invasive access to the implantation site and its preparation. The compression of the implant is maintained either directly by projections 34 on the inner contour of the sleeve or by a compression clasp 35. After the implant has been placed in its optimum position between the spinous processes, the implant unfolds to its intended size through withdrawal of the sleeve 33 or the compression clasp 35.

FIG. 7 shows a variant 200 of the implant 20 with a ventral stabilisation clip 220. For attaching to the ventral aspect 250 of the implant 20 there are suitable projections 211 and/or recesses 212 at the transition to the implant shanks 252, 256,

The stabilisation clip 220 can vary in thickness. Thus, the middle section can be thicker (D1) than the ends (D2). The width b of the stabilisation clip 220 can also vary in its longitudinal direction. Said variants of the stabilisation clip 220 are aimed at stabilising the ventral aspect 250 of the implant 20 but without essentially increasing the angular stability, i.e. the stability in the area of the ends.

FIG. 7A shows axial spurs 222 on the axial ends of the stabilisation clip 220 in order to prevent lateral slippage or migration of the clip 220. The spurs 222 engage in corresponding holders within the recess 212 or project above the recesses 212 on the external surfaces of the implant.

FIG. 7B shows an alternative possibility for securing the stabilisation clip 220 against lateral migration. Here the stabilisation clip 220 has tooth recesses 224 into which corresponding toothed projections 226 with the implant recesses 212 engage.

FIG. 8 shows a further variant 300 of the implant 20 in which the clip-like shoe 358 forms an arch 340 running perpendicularly to the longitudinal axis of the shank 354 and the two ends of which 360 are both radial deformed inward as a result of which teeth are formed. This allows flexible fitting to the spinous process 4 and attachment thereto through deformed ends 360.

In order to further optimise fitting to the surface of a spinous process 4 varying in width, the sides of the clip-like shoe 358 can be designed like a tongue through slit 320. The tongues 330 can thus individually be supported on the spinous process 4.

FIG. 9 shows a variant 400 of the implant 20 in accordance with FIG. 8 in which the clip-like shoe 458 is only combined with the remainder of the implant later during the production process. This allows a combination to two materials which exhibit the properties for the relevant requirements. Thus, the ventral aspect 410 can be made of a spring elastic material, while the slip-like shoe 458 can, for example, be made of a deformable material or of a memory metal (nitinol). This allows bending and insertion of the ends 460 into the spinous process 4 (e.g. with the aid of tongs). When using a memory metal the clip-like shoe 460 can be designed so that it is opened wide during implantation and after implantation the shoe 460 is heated. The cheek 440 bend in a arch-like manner toward the spinous process 4 and with the ends 458 attach the implant 20 to the spinous process 4.

To facilitate lateral insertion the saddle can also consist of any one of the aforementioned materials. If it is made of a memory metal, the opening angle of the saddle on insertion would be around 150°-180° (i.e. practically flat), and after slight heating it would adjust to the previously described values (of the desired shape) for the saddle-like support.

The U-shaped stirrup 410 is permanently connected to the clip-like shoe 458 by means, for example, of a welding seam, an adhesives, screw, rivet or snap-type connection 450.

Accordingly the saddle-like support 156 can be manufactured separately and connected with the U-shaped stirrup before or during the operation. In this way the stirrup can, for example, be made of a simple, curved metal strip or a fibre composite material, and the supports and shoes, possibly after selecting the corresponding anatomical shape and breadth, can engage in or be connected to the ends of the shanks in another way.

FIG. 10 shows a dorsal view of an assembled further two-part variant 500 of the implant 20. In this variant 500, which is shown in an exploded view in FIG. 10A, the clip-like shoe 558 is detached from the stirrup 510. On the implant 20 there are lateral recesses 540 on one shank 554, which internally hold or are covered by the clip-like shoe 558. In this way the implant 20 can be inserted laterally between the spinous processes 4 and 5 without of with only minor compression. One inserted the shoe 558 for fixing to the spinous process 4 is also inserted laterally and then pushed via the implant shank 554 onto the spinous process 4. For fixing to the implant shank 554 there are appropriate engaging mechanisms, e.g. tongues 530 inwardly notched out of the side sections of the shoe 558. The sides of the clip-like shoe 558 are either spring elastic so that they are optimally supported on the spinous process 4, or are subsequently bent to the width of the spinous process. On the inner surface of the side sections of the shoe 558 there are surface modifications 560, for example teeth, for fixing to the spinous process 4.

FIG. 11 shows a dorsal view of a three-part variant 600 of the implant. The implant 20 has a curved shoed 658, the ends 660 of which are pointed and rest on/are anchored in the spinous process 4. The saddle-like support 656 for the spinous process 5 can also be seen on the opposite side of the implant 20. The shoe 658 has a support 640 which is provided with at least one snap-type connection 642 which passes through a corresponding recess in the shoe 658 and the corresponding shank of the stirrup 610. The barbed hook-like design of the snap-type connection 642 connects the shoe 658 to the U-shaped stirrup 610 after engaging. On implantation the curved shoe 658 is initially inserted and fastened to the spinous process 4 with the support 640. The slightly compressed stirrup 10 is then inserted laterally, positioned and finally snapped into place on the clip-like holder of the support 640 via the snap-type connections 642.

FIG. 12 show a further variant 700 of the implant 20, which has a retaining strap 730 for fixing to the spinous process 4. The implant shank 754 is saddle-shaped 755 to fit optimally around the spinous process 4. At locking device 720 is attached to the shank 754 on which on one side the start of the retaining strap 730 attached and on the other side there is a fastening mechanism 760 which takes up the retaining strap 730 after it has been passed around the spinous process 4. This locking device 720 can be produced separately, or be firmly integrated into the shank 754. To facilitate threading of the end 734 of the strap 730 into the locking device 720 the retaining strap 730 is preferably stiffened at the end 734. The projecting end 734 can be cut off after positioning, tightening and locking by the fastening mechanism 760.

On the shanks 752 and 754 there are within the support surfaces suitable surface modifications 762 and 764, for example, teeth, which prevent migration of the implant 20 and thereby stabilise the spinal segment 1 in the dorsoventral direction.

FIG. 13 shows a dorsal view of variant 700 of the implant in accordance with FIG. 12. FIGS. 13A, 13B and 14C show dorsal sectional views of examples of retaining strap 730 fastening variants.

In FIG. 13A variant 770 of the fastening mechanism 760 has a wedge 774, which after threading and tightening of the retaining strap 730 fixes the latter. The wedge 774 is connected with a pin 772 of the locking device 720 to prevent it falling out. One tightening of the retaining strap 730 the free end 734 is wound up with an instrument (e.g. a needle holder) in a rotary movement whereby, if the wedge 774 is suitably designed, it is also pressed into the device 720 and thereby secures the retaining strap 730 against slipping out.

FIG. 13B shows an eccentric fastening mechanism 780 which also allows fixing of the retaining strap 730. The fastening mechanism 780 has a disk 784, which can be round or oval, and is preferably eccentrically 782 attached to the locking device 720. The disk 784 can also be held by a spring in the tensioned position (arrow), which only allows the retaining strap 730 to be pulled through in one direction. The eccentric disk 782 can however also be fixed externally in the tightened position by means of a rotary movement (with a screwdriver for instance).

FIG. 13C shows a fastening mechanism 790 which through a snap-lock 794, similar to a cable binder, only allows the retaining strap 730, which is provided with teeth for this purpose, to be pulled through in one direction thereby fixing it in this position after tightening. The snap-lock 794 made of a spring leaf can elastically move into a free space 793 when the retaining strap 730 is being pulled through, whereas in the opposite direct is only has a small room for manoeuvre against a stop 792. Through the oblique position vis-à-vis the retaining strap 730 the snap-lock 794 is pressed against it when pulling back the retaining strap 730 until the stop 792 blocks the movement of the snap-lock 794.

In the case of spinal column segments 1 which already exhibit a high degree of hypermobility, a derivation of the implant 20 in FIGS. 12 and 13 is useful in order to also restrict the flexion movement. For this lifting of the saddle-like support 752 from the spinous process 5 is prevented in that a second device 720 is connected to the implant shank 752 so that two adjacent spinous processes 4 and 5 are each connected by a retaining strap 730 to the spring elastic stirrup 710.

In FIG. 14 variants a) to e) of the horse saddle-like support 156 are shown. The saddle-like support 156 can be produced as a separate component or integrated into the shape of the stirrup 10. As described above, the saddle shape is orientated towards the shape of the spinous process 5 with the aim of providing as flat support as possible. The implant variants shown, which allow lifting of the saddle-like support, are preferably saddle shaped 14 a) to e). Other variants, in which fixing to the spinous process 5 is to take place, are preferably designed with a saddle shape in accordance with FIG. 14e).

FIG. 15 shows a further variant 800 of the implant 20 with a projection 855 under the upper shoe 158, which as shown in FIG. 15A prevents the loop/retaining strap around the spinous process 4 slipping on the implant.

FIG. 16 shows a fastening variant of the implant 800 in which the retaining strap 860 is passed through a sleeve 870.

FIG. 16A shows the implant after insertion between the spinous processes 4 and 5, whereby the sleeve 870 was previously implanted into the spinous process. This anchoring in the spinous process allows securing fixing and the sleeve prevents the retaining strap from cutting into the bones.

FIGS. 17 and 17A shows a further loop/retaining strap fastening variant 955 on variant 900 of the implant 20. Above the upper shoe 158 there is a projection 955 to which the loops/retaining straps for fixing the implant to the spinous process or vertebral arch can be attached.

In order to prevent ventral migration of the implant, projections 959 are also envisaged behind the shoe 158, which after implantation are in contact with the vertebral arch.

FIG. 17B shows implant 900 after insertion between the spinous processes 4 and 5 and fastening with loops 960, which are passed round the vertebral arch in a sublaminar manner.

A variant 1000 of the implant 20 is shown in FIGS. 18, 18A and 18B. Wings 1055 located laterally on the shoe 158 are supported on the vertebral arch and can be fixed with clips 1060 or loops.

FIG. 18 shows one possible shape of the clips 1060. They surround the vertebral arch laterally from the spinous process and are adjustable in length. In the tensioned state they attached the wing firmly to the vertebral arch.

FIG. 19 shows modifications 1151 and 1152 of a variant 1100 of the implant 20 on the support surfaces of the lamina of the relevant vertebral arch. At these points a softer material is applied, e.g. silicone or PCU, to counter irritation of the lamina. The support modification can, as shown, only affect the lamina area, or can optionally also be present in the area of the shoe 158 and/or the saddle-like support 156.

FIGS. 20, 20A, 20B and 20C show further variants 1200 of the implant 20 in which the ventral aspect is curved in three places. In this way the stability of the implant can be optimised and the compressibility necessary for lateral insertion can be improved. FIGS. 20 and 20 shows projections 1255 located laterally on the shoe 148 which are used to fix the implant by means of loops.

FIG. 20B shows an variant of the implant without the projections 1255.

In FIG. 20C the projections 1295 are like wings and rest against the vertebral arch after implantation. They can be fixed to the vertebral arch with clips of loops/retaining straps.

FIG. 21 shows another variant 1300 of the implant 20 which can be used in the area of the last lumber vertebra—first sacral vertebra and has paddle-like spurs 1355. The spurs 1355 can have surface modifications in the form of spikes, holes, coatings or groove which allow for better anchoring. For fixing the implant they are pushed laterocaudally of the spinous process of the last lumbar vertebra between the tendons located there, or between the tendons and sacrum.

As can be seen in FIG. 21A the upper shoe 1358 is saddle-shaped and the saddle-like support 1356 modified so that it can optimally be supported on the spinous process of the first sacral vertebra. The spurs 1355 can vary in shape and number and only be connected to the implant on a suitable device 1357 during or after implantation.

FIG. 21B shows the implant 1300 after insertion between the spinous process 1304 of the last lumber vertebra and the spinous process 1305 of the first sacral vertebra. The paddle-like spurs 1355 are pushed between the sacrum 1307 and the tendons 1306 attached to it.

In FIG. 22 a further variant 1400 of the implant for the area last lumbar vertebra—first sacral vertebra is shown. The upper shoe 1458 for holding the spinous process is saddle-shaped, the lower shoe 1456 has a deeper incision to hold the first sacral vertebra better and to take into account the different anatomical conditions.

FIG. 22A shows the implant 1400 with an M-shaped constriction 1455 on the implant shank 1452, which is intended to improve support on the lamina of the first sacral vertebra. In order to be able to attach the implant to the sacrum using loops/retaining straps or clips, it has a recess 1451 through which the loops can be passed of the clips engaged.

FIG. 22B show loops 1460 passed through the recess 1451.

In FIG. 22C the implant is inserted between the spinous processes of the last lumbar vertebra 1404 and the first sacral vertebra 1405. With the aid of clips 1470, which each engage on the foramen sacrale dorsale 1407, the implant is fixed to the sacrum 1406.

FIG. 23 shows a variant 1500 of the implant 20 for area last lumber vertebra—first sacral vertebra. The implant has lateral projections 1555 and 1545 which allow fastening of the implant to both the last lumbar vertebra and the sacrum.

As can be seen in FIG. 23A the upper shoe 1558 is designed with double paddle in order to be supported on the spinous process. Behind the paddles there are further projections 1559 which are supported on the ventral arch and prevent migration to ventral.

In FIG. 24 a further variant 1600 of the implant 20 is shown. The ventral aspect 50 is thickened in a block-like manner in order to achieve the necessary stability for restricting extension movement of the vertebral segment when softer materials are used. To preserve mobility in flexion the ventral aspect is connected via flexible shanks 1654 and 1652 to the supports 1656 and 1658. These can be connected to the spinous processes by means of loops/retaining straps or clips. In order to be able to be used in the area last lumbar vertebra—first sacral vertebra the saddle-like support 1656 can be broadened and the shank 1652 lengthened, and the angle between the ventral aspect 1650 and shank 1652 adjusted.

FIG. 24A shows a perspective view of the implant 1600. Behind the shoe 1658 there are spurs 1659 which after implantation are supported on the vertebral arch. For supporting on the lamina the ventral aspect 50 can have a step 1659.

FIG. 24B shows a modification 1670 in the area of the implant shank 1652 in the form of a constriction in order to increase flexibility. The implant can thus better follow the flexion movement of the vertebral segment. This constriction can be present in one or both implant shanks.

In FIGS. 25 and 25A a further implant variant 1700 can be seem. The implant comprises a two-part shell 1750 which is connected to support surfaces 1758 and 1756 for the spinous processes. Aspect 1750 comprises a double shell 1741 in which one shell 1740 slides. For this the double shell 1741 has sliding surfaces 1751 and 1752 facing each other. Within these sliding surfaces shell 1740 slides, which also has sliding surfaces 1753 facing the double shell 1741. By modifying the shell edge complete separation of the two parts of the implant can be prevented or the sliding movement restricted to a desired extent.

FIG. 25B shows the curvature of the shells in the sagittal plan. The radii of all the sliding surfaces are concentric to each other and meet at the physiological centre of rotation of the vertebral segment, which is at a distance from the relevant shell with radius 1755.

FIG. 25 C shows the shell curvature of the shell 1750 in the transverse plane. The radii of all the sliding surfaces are concentric to each other and meet at the physiological centre of rotation of the vertebral segment, which is at a distance from the relevant shell with radius 1754.

FIGS. 26A and 26B show a further development of the implant 1700 described in FIG. 25 in that a strand of elastic material 1710 (e.g. silicone, PCU) is inserted between the double shell 1741. For lateral insertion it may be advantageous not to insert the strand until after implantation of the two-part implant and thereby restrict the extension path of the vertebral segment.

FIGS. 26C, 26D and 25E show various view of the implant 1700 after implantation. FIG. 26C shows a dorsal view, FIG. 26D a perspective view and FIG. 26E a lateral view with the centre of rotation Dz drawn in.

1. Spinal column implant (20) in veterinary or human medicine for the dynamic stabilisation of spinal column segments (1) which can be placed between two spinous processes (4, 5), with a stirrup (10), having ventral aspect (50), the free ends of which in the direction of the spinous processes (4, 5) are provided with means (11, 12) for supporting on the spinous processes (4, 5), characterised in that the width of the implant can be reduced for implantation so that the implant width including the means (11, 12) corresponds approximately to nominal implant width B and that at least one of the means (11, 12) is designed as a saddle-like support (56, 156), the saddle flap of which is so short that the implant can be inserted laterally between the spinous processes.

2. Spinal column implant in accordance with claim 1 characterised in that the means have shanks (13, 14) and at least one of the shanks (13, 14) is provided with an edge-open incision or drilled hole (170) for the attachment and slip-resistant use of a compression instrument (30).

3. Spinal column implant in accordance with claim 1 characterised in that the implant (20) can be implanted via an implantation sleeve (33) so that minimally invasive access is possible.

4. Spinal column implant in accordance with any one of the previous claims characterised in that one of the means (156) is provided at least in sections with a surface modification facing the spinous process (5) in the form of riffling, coating, grooves or toothing (162, 164) to prevent dorsoventral migration (displacement of the implant in the dorsoventral direction).

5. Spinal column implant in accordance with any one of the preceding claims characterised in that the second means (11) are designed as a spring-elastic retaining clip (358, 458, 658) which is spread open by the spinous process and thereby rests on the spinous process in a pre-tensioned manner.

6. Spinal column implant in accordance with any one of the preceding claims characterised in that the first and/or second means (12 and/or 11) are formed of a retaining module (558), fixable on the free end of the shank (152, 154) and in contact with the spinous process (4 and/or 5), which after implantation of the stirrup (10) can be attached thereto.

7. Spinal column implant in accordance with any one of the preceding claims characterised in that along its ventral aspect (150, 250), the stirrup (10) can be provided on its inner site with inserts (220) of various curvatures and/or spring rates after implantation in order to increase its compression stability.

8. Spinal column implant in accordance with any one of the preceding claims characterised in that the ventral aspect is (250) is curved three times.

9. Spinal column implant in accordance with any one of the preceding claims characterised in that the first and/or second means (11, 12) are formed by a loop or a retaining strap (730) surrounding the spinous process (4) which can be adjusted in width and affixed, and this retaining strap can be passed around the spinous process, or after boring through the spinous process and insertion of sleeve (870), threaded through the spinous process.

10. Spinal column implant (1300) in veterinary or human medicine, more particularly for stabilising spinal column segments, which can be placed in the area of the last lumbar vertebra (1304) and the first sacral vertebra and is provided with means (1356, 1358) for supporting on the spinous processes (1304, 1305), characterised in that for fixing lateral spurs (1355) are attached to the implant which after prior preparation are inserted into or under the aponeurosis on the sacrum.

11. Spinal column implant in accordance with any one of the preceding claims characterised in that the lateral spurs (1355) used for fixing the implant are only connected thereto during or after inserting the implant.

12. Spinal column implant in accordance with any one of the preceding claims characterised in that the lateral spurs (1355) used for fixing the implant are provided with suitable surface modifications (1364) such as roughening, grooves, holes or spikes.

13. Spinal column implant in accordance with any one of the preceding claims characterised in that for better contact on the lamina of the sacral vertebra, for the area last lumbar vertebra-first sacral vertebra the implant has a bowed narrowing (1455) and can be fixed by means of loops (1460) or clips (1470) to the first sacral vertebra spinous process or the forr. sacralia dorsalia.

14. Spinal column implant in accordance with any one of the preceding claims characterised in that the ventral aspect 50 is designed in the form or a block and the ventral aspect is flexibly connected via two shanks (1652 and 1654) to holding means (1656 and 1658).

15. Spinal column implant in accordance with any one of the preceding claims characterised in that at least one shank (1652) has a constriction (1670) to increase flexibility.

16. Spinal column implant (1700) in veterinary or human medicine, more particularly for stabilising spinal column segments, which can be placed between two spinous processes (4, 5), with a shell (1750) provided with means (1756, 1758) for supporting on the spinous processes (4, 5), characterised in that the shell (1750) is curved dorsally, the curvature is orientated to the pivot point of the vertebra segment (1754, 1755) and the shell (1750) is in two parts.

17. Spinal column implant in accordance with claim 16 characterised in that at least one strand (1710) of an elastic material is arranged between the two halves of the two-part shell (1750).

18. Spinal column implant in accordance with any one of the preceding claims characterised in that the implant has projections (855, 955) which allow the fixing of loops/retaining straps (860) on the implant, whereby the loops are passed in a sublaminar fashion around the vertebral arch or around the spinous process.

19. Spinal column implant in accordance with any one of the preceding claims characterised in that the implant has wings (1055) which can be affixed to the vertebral arch with clips (1060) or retaining straps (960).

20. Spinal column implant in accordance with claim 17 characterised in that the implant has lateral spurs (1545, 1555) which are used for fixing by means of clips or loops.

21. Spinal column implant in accordance with any one of the preceding claims characterised in that the contact surface (1151, 1152) for the laminas and/or the spinous process is made of a second, softer material for cushioning.

22. Spinal column implant in accordance with any one of the preceding claims characterised in that the first means (12), the second means (11) and/or the ventral aspect (50, 250) of the implant is made of a highly elastic material: nitanol, titanium or titanium alloy, spring or implant steel, a cobalt-chromium alloy such as CoCr, CoCrMo, CoCrNiMo, polyethelene, polycarbonate urethane, PEEK, PEK, PEKK, PSU, PPS or silicon as well as a fibre composite material or a combination of several or these materials.

23. Spinal column implant in accordance with any one of the preceding claims characterised in that the first and/or second means (12 and/or 11) correspond in shape and/or width to the anatomical shape of the spinous process (4, 5).
